(19) United States
(12) Patent Application Publication
(10) Pub. No.: US 2011/0276312 A1
(43) Pub. Date: Nov. 10, 2011

(54) DEVICE FOR MONITORING AND MODIFYING EATING BEHAVIOR

(76) Inventors: Tadmor Shalon, Palo Alto, CA (US); Leonardo Gabriel Neumeyer, Palo Alto, CA (US)

(21) Appl. No.: 12/601,415

(22) PCT Filed: Jun. 2, 2008
(86) PCT No.: PCT/IL08/00742

§ 371 Date: Jun. 16, 2010

Related U.S. Application Data
(60) Provisional application No. 60/929,008, filed on Jun. 8, 2007.

(51) Int. Cl. G06F 17/40; G06F 15/00; G01P 15/00; A61B 5/00

(52) U.S. Cl. 702/187; 73/865.4; 702/189; 73/488

(57) ABSTRACT

A device for monitoring eating behavior of a user is provided. The device includes at least one sensor mounted on a head of the user, the sensor being capable of detecting jaw muscle movement and sound while not occluding an ear canal of the user.

### FIELD AND BACKGROUND OF THE INVENTION

The present invention relates to systems and methods for monitoring and modifying eating behavior of a subject such as a human or an animal.

Successful dieting requires long-term behavior modification in terms of eating and physical activity. A diet plan is only part of the solution. Sticking to the plan requires behavior modification that is generally beyond the ability of most people to implement without external assistance. There does not appear to be a diet or process by which people can reliably lose weight and keep it off. Numerous studies have shown 100% weight regain on most diets. Other meta-studies show that people regain approximately 75% of the initial weight loss after five years.

Assessing energy balance, i.e. the net difference between energy intake and expenditure is central to obesity research, prevention, and treatment. The importance of accurately measuring energy balance is appreciated by considering the dynamics of average weight gain in middle aged adults which is about 10 lbs. per decade. This significant gain in weight follows from a net intake excess of approximately 0.3% of the daily calorie consumption, which is below the awareness of most individuals. [National Institutes of Health, Bioengineering Approaches To Energy Balance And Obesity (SBIR/STTR) grants1.nih.gov/grants/guide/pa-files/PA-04-156.html].

Today, energy intake is at best only crudely measured by self reporting food consumed, an approach that nutritionists know falls well short of its accuracy goals. Although standard self-report questionnaire and recall techniques can provide valuable data on dietary patterns, these techniques are time consuming, inconvenient, and infamous for considerable underreporting of food consumed, with this error more pronounced for over weight than non-over weight individuals.

Several devices and methods which attempt to overcome the deficiencies of self reporting approaches have been described in the prior art.

U.S. Pat. No. 6,135,950 describes a pager size device to aid in controlling a person's daily food intake. U.S. Pat. No. 5,398,688 describes a timer for calculating and alerting a user when their maximum eating time has expired. U.S. Pat. Nos. 5,188,104 and 5,263,480 describe the treatment of eating disorders by nerve stimulation by detecting preselected events indicative of imminent need for treatment and applying predetermined stimulating signal to patient vagus nerve. PCT Publication WO 02/053093 and US Application Publication No. 2004/0147816, describe a similar invasive technique except that the stimulation is driven into the stomach muscle of the subject, thereby altering the timing of digestion. PCT Publication No. WO 02/026101, describes a generic arrangement of implantable sensors, microprocessors and a negative-feedback stimulator which can enforce a corrective regimen on a patient suffering from a dietary or other behavioral disorder. U.S. Pat. No. 5,263,491 describes an ambulatory metabolic monitoring unit which employs a mastication microphone to estimate food intake and an accelerometer to detect movement. JP applications 11318862 and 10011560 describe in-ear devices for monitoring mastication and sound.

Unfortunately each of the approaches described above suffers from inherent limitations that prevent use thereof as an eating behavior modification device. The only non-drug interventions for losing weight that display some long-term efficacy are the various procedures to reduce the volume of the stomach or bypass it altogether so that just a small volume of food may satiate the patient. While such approaches show some promise, they require invasive surgical procedures with attendant risks and pain, they often require permanent prosthetic implants and/or irreversible modification of the patient's digestive tract with potentially serious complications and side effects, they are costly, and they require long recovery time during which the patient is immobile and unproductive. Many of those who are overweight or obese are thus unable or unwilling to undergo such interventions.

There is thus a widely recognized need for, and it would be highly advantageous to have, a device for accurately monitoring and controlling eating behavior of a subject without the invasiveness, risks, pain, complications, cost, and recovery time associated with stomach volume reduction and bypass procedures.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 a flow chart illustrating processing of sensor data by the device of the present invention and extraction of eating and physical activity signals.

FIGS. 2a-d illustrate the components (FIG. 2a), an in-ear configuration which includes a neck mounted accelerometer (FIG. 2b), a combined behind the ear in-ear configuration (FIG. 2c), and in-ear configuration (FIG. 2d) of the present device.

FIG. 3 illustrates ear anatomy.

FIG. 4 images showing skull muscle locations without (top image) and with (bottom image) reference to ear skull location.

FIGS. 5-6 are graphs illustrating daily eating times of a man (FIG. 5) and a woman (FIG. 6) as monitored by the device of the present invention.

FIG. 7 is a flow chart diagram illustrating sound capturing and processing as conducted by the present device.

FIG. 8 illustrates a typical signal that has a speech segment followed by two chews. Arrows 1-4 indicate the four phases of a chew cycle.

FIG. 9 illustrates extraction of the low frequency component of chewing; The top panel (panel A) illustrates a signal generated by speech followed by chewing, the signal is then rectified (panel B), low pass filtered (panel C) and segmented (panel D).

### DESCRIPTION OF THE PREFERRED EMBODIMENTS

The present invention is of a device which can be used to monitor and optionally modify an eating behavior of a user.

The principles and operation of the present invention may be better understood with reference to the drawings and accompanying descriptions.

Before explaining at least one embodiment of the invention in detail, it is to be understood that the invention is not limited in its application to the details set forth in the following description and accompanying Examples. The invention is capable of other embodiments or of being practiced or carried out in various ways. Also, it is to be understood that the phraseology and terminology employed herein is for the purpose of description and should not be regarded as limiting.

Numerous approaches for modifying eating habits are known in the art. Although some benefits can be gained from using such approaches, none provide an ideal diet control solution as is evidenced by the growing need for new solutions for treating eating disorders such as obesity or anorexia.

Although prior art devices which utilize throat-mounted or in-ear sensors for detecting sounds and eating-related jaw movement have been described (see, for example JP applications 11318862 and 10011560 or U.S. Pat. No. 5,263,491), such devices suffer from limitations related to ease and comfort of use and sound processing for extraction of eating-related sound activity and thus cannot be effectively utilized to provide accurate monitoring and feedback to a user.

While reducing the present invention to practice, the present inventors realized that an effective eating behavior-modifying device that results in sustained and persistent weight loss must take into consideration the following:

(i) Convenience—a big risk for efficacy and consumer adoption is the practicality of wearing the device daily throughout the weight loss period (on average 20 weeks) and thereafter for weight maintenance. Taping sensors to the skin, implants, social stigma associated with highly visible elements negatively impact consumer adoption and usage. A device positioned on the throat or trunk is undesirable; wires connecting elements of the system should not be visible or obtrusive. It should be easy for the user to deploy or remove and should be minimally disruptive or noticed throughout the day (like a pair of glasses).

Thus, the present device should be as small as possible and hence power efficient since the battery volume required for a day or more usage dominates the device size. It should require minimal input from the user and automatically adjust itself to the user behavior. User control over the device while worn should preferably be implemented by vocal or acoustic commands hence eliminating other traditional user interface mechanisms (buttons, screens, etc.).

(ii) Accuracy—in order to determine small changes of less then 10% of total caloric input or ingestion time (as proxy for the former for a particular user) the device must be able to take measurements in the field under various conditions including speech by user or others, background noises, physical activities such as walking, for all types of food. It must be able to accurately distinguish jaw movements associated with chewing from those made for other reasons such as talking, drinking or swallowing of saliva.

(iii) Feedback—the device should provide discrete motivational feedback and yet be unobtrusive to the user (e.g., muted when user is speaking) and sensitive to user environment (e.g., background noise level).

(iv) Social context—features enabling social context are important in both the efficacy aspect of the device and compliance and thus the device should be provided with networking capabilities preferably by being capable of interfacing with a networking-capable device such as a cellular telephone or a computer.

(v) Usage and Compliance—The device and related system should be able to determine whether the user is using the device as needed and whether he or she complies with feedback.

Thus, according to one aspect of the present invention there is provided a device for monitoring and optionally modifying an eating behavior of a user. The terms “user” and “subject” are interchangeably used herein to refer to a mammal, preferably a human in need of eating behavior modification.

The device of the present invention includes three sub-systems, a sound, jaw activity, body movement detection subsystem; a data gathering/processing subsystem; and a feedback transducer (e.g. miniature speaker). All of these subsystems could be integrated into a single enclosure that is worn on the head of the user and is preferably operated by the user via voice or acoustic commands.

The sensors are selected capable of sensing mechanical (e.g. jaw motion) and sense the acoustic signals created by the user's activities. The sensors are mounted in a convenient, (single site) location on the head. In-ear canal mounting should be effected in a manner that does not occlude ear canal, since ear canal occlusion can impair hearing and lead to user discomfort especially over extended periods of use and hence affect the subject's usage and compliance. Prior art devices (see JP patent applications ibid) require a seal in order to measure pressure in the ear canal, in addition to the problems mentioned above, such an approach will unknot likely work for extended time periods because it will be hard to maintain a seal without causing ischemia and hence pain in the surrounding tissues.

The device can include a head mounted sensor for sensing sounds and jaw movement and one or more additional sensors that are preferably mounted on the head or elsewhere and can sense movement, ambient sounds etc.

The device preferably includes a unified sensor array which enables simultaneous measurement of two or more modalities thus enabling isolation of eating sounds from non-eating sounds (ambient sounds, vocal chord-emitted sounds etc).

Correlation of at least two modalities (e.g. sound and jaw movement) enables accurate detection of mastication events and very low false positives/negatives rates (jaw moves during speech—non-eating related mastication, etc). In addition, correlation of jaw movement and environmental sounds that can interfere with the measurements can also be used to filter our non-eating related sounds.

A microphone which can be coupled to the cranium or connected bones in order to pick up sounds associated with food ingestion and mastication, can be, for example, a conduction microphone such as a piezo film microphone pressed against the skin. The microphone can be A Sonion 9721 [Sonion Inc.] which is modified for low frequency response) or a MEMs fabricated microphone array.

A sensor capable of detecting the deformation of tissue resulting from the change in tissue (e.g. muscle) volume due to mastication and food ingestion can be a piezo film pressed against the skin, or a suitable air tight bubble which can be incorporated into the microphone described above. The bubble and microphone can act as a pressure transducer and record changes in pressure within the bubble due to changes in the topology of the contacted skin.

An open air microphone mounted near the bone conduction microphone but able to detect environmental sounds could be utilized to subtract ambient sounds from the sounds detected by the bone conduction microphone in order to further improve signal/noise ratio and record user voice commands.

In a preferred configuration, the present device includes in a single housing a microphone with special acoustic impedance matching element (e.g., bone conduction microphone capable of frequency response to 0.1 Hz) capable of picking up high frequency sounds conducted by the cranial bones and anatomical deformation caused by mastication and manifested as subsonic pressure signal (0.1 Hz-20 Hz) and thus detect anatomical deformation caused by jaw movement. For example, a microphone having a bubble such as NextLink InVisio with a modified microphone (with low frequency response) can act both as an effective bone conduction microphone capable of broad spectrum frequency pickup (down to 0.1 Hz) with high rejection of background acoustic noise, as well as a pressure transducer capable of detecting slight deformation of the bubble due to changes in surface anatomy caused by mastication.

The mounting location of such a microphone/pressure sensor is critical for both convenience and operability. Head surface regions having an area of cm² or less and being a junction of acoustic energy and mastication muscles induced topical deformation are preferably used as mounting locations. Examples of preferred location include the entrance to the external ear canal (without occluding the canal) or above, behind or below the ear (see Example 1 below for additional detail). Such locations allow the user to wear the device discretely while enabling user communication through a microphone and speaker as is described herein.

Other sensor combination can include a microphone to pick up cranial sounds and piezo film or accelerometer to pick up local skin deformation.

The device of the present invention can utilize an additional sensor (e.g. accelerometer) for measuring body movement and correlating such movement with jaw movement as part of the sensor array described above. Such an accelerometer can also be used to collect information regarding physical activity of the user and thus energy expenditure as well as be correlated with the microphone and deformation sensor to filter our movement related signals which might be misconstrued as eating-related activities.

The present device can also include an additional microphone to pick up environmental sounds that to be subtracted from the sounds acquired by the first microphone. Preferably, the head-mounted bone conducting microphone described above can be configured having a port exposing the diaphragm to environmental sounds.

The device also incorporates a sound generating transducer such as a speaker for relaying feedback to the user regarding eating activity and/or for coaching the user. Alternatively, a separate speaker device can be mounted in the housing of the present device.

The present device can be powered using an internal power source such as a battery, rechargeable battery, a large capacitor, a fuel cell, or extract power from the temperature differential between the body and the surrounding air such as described by way of example in U.S. Pat. No. 6,640,137 Biothermal Power Source for Implantable Systems or extract power from the motion of the user such as the mechanism in the Seiko Kinetic watch.

The present device can be powered from a connection to an external device such as a cellular phone, an MP3 player or a PDA. The sensor(s) can be charged by inductance by simply placing the device in proximity to an electrical potential. The sensors can be powered by a coil and rectifier that generate DC power from transmitted RF signals generated by a charger coil as is well known in the art of implanted medical devices.

Acoustic energy generated by chewing, swallowing, biting, sipping, drinking, teeth grinding, teeth clicking, tongue clicking, tongue movement, jaw muscles or jaw bone movement, spitting, clearing of the throat, coughing, sneezing, snoring, breathing, tooth brushing, smoking, screaming, user's voice or speech, other user generated sounds, and ambient noises in the user's immediate surroundings can be monitored through one or more sensors (e.g. microphones) as described above.

The sensor(s) transmits acquired sound information to a processing unit contained in the device of the present invention.

The sensor may transmit such information continuously (100% duty cycle) or only when an event is detected (described below). When not transmitting sounds, the microphone and the associated electronics can be in standby mode to conserve power.

The processing unit of the present device is capable of processing the data sensed by the sensor(s) and deriving eating-related activities therefrom.

Such processing enables qualification and quantification of eating behavior and thus enables real-time monitoring. When such monitoring is combined with personalized and historical data in an expert system, an appropriate feedback can be given to the user in order to modify his/her eating behavior.

The device could distinguish between different foods by the sound made during their mastication and how the user is chewing them (rate, strength, number of chews) or by user input (e.g., by speaking the name of the food or selecting it from a spoken menu of options). The caloric density of each food type can be stored in the device's memory. The device could further infer volume of the food by how long it has been chewed and calibrating such measurement to each individual user. By multiplying these two quantities, an estimate of caloric input is attained.

However, with most people food choices remain relatively constant when averaged over a day or a week and hence most people do not gain or lose significant weight over such period. Hence, a strategy of portion control can be employed by the device where the assumption is made that the caloric density of the food is relatively constant over a period of days or a week and the object is to require the user to eat less of whatever baseline diet they are on. Hence a 10% random reduction in ingestion will lead to 10% overall reduction in consumed calories for a given user and hence to the corresponding weight loss. If the device also monitors the user's weight (via entered, spoken or scale with Bluetooth or other electronic means of communications), any changes of baseline diet or changes in caloric output can be monitored periodically against the desired eating time budget. If the anticipated weight loss was not achieved, the baseline is adjusted downward in the subsequent period correspondingly.

The processing unit includes memory for storing executable software components which incorporate one or more algorithms for processing and correlating input obtained from one or more sensors.

In order to have the most convenient user deployment of the sensors and low power requirements, a small processing package including sophisticated, computationally efficient algorithms are employed and CPU whose clock (and hence power consumption) can be algorithmically controlled by the software depending on its computational needs. Computationally complex algorithms such as the Fast Fourier Transform (FFT) are implemented in fixed-point arithmetic which can be run efficiently with minimal power consumption on a standard CPU with a fixed-point arithmetic unit.

The food ingestion detection algorithms employ the following elements:

(i) An accelerometer processing algorithm which determines whether there is gross body movements such as gate in order to avoid to have to process the signal from other sensors.

(ii) A computationally efficient speech detector (software or in ASIC) in order to determine if jaw movements are caused by speech (this information is also used in the user interface)

(iii) A chew detector monitoring jaw movement detector (the anatomical surface deformation detector described above mounted near the jaw and related muscles) and by input from other detection algorithms and the context able to classify chew events highly accurately.

(iv) Eating sound detection and classification (statistical algorithms). Processes the information retrieved from the sensors and increases the accuracy of the chew detector algorithm; also allows for food type recognition.

(vi) Speech analysis algorithm—a part of the user interface, operates only when speech is detected by (i) thus conserving power.

(vii) A swallow detector which infers swallowing from the nature of the chews energy content and timing which is used in subsequent ‘lap band’ and food volume estimation algorithms.

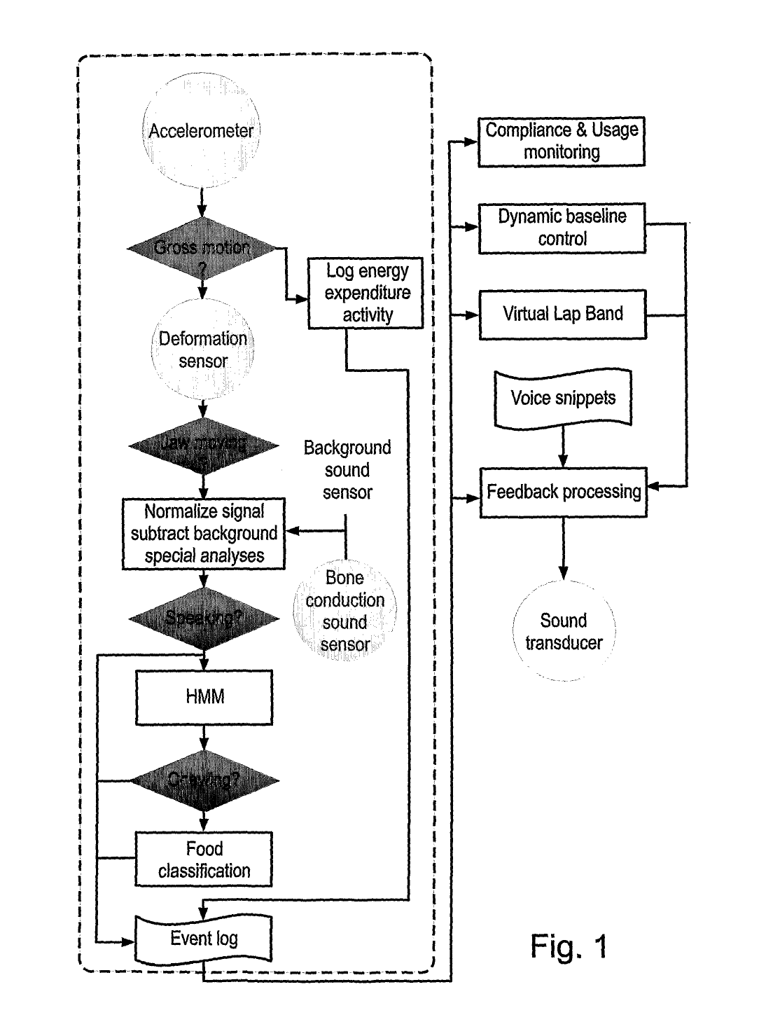

FIG. 1 is a flow chart illustrating detection and processing of sounds as effected by the present device. Further description of sound analysis is provided in Example 3 of the examples sections which follows.

The information retrieved by the sensors and processed using the above described algorithms can be used to generate information related to eating habits of the user as well as physical activity thereof.

Signals from the sensors are coupled via a low power signal conditioning circuitry such as pre amp with linear or log gain to a broad frequency (down to DC) network to the analog to digital converter (A/D). Multi-channel or multiplexed A/D samples the sensors under software control to minimize power consumption (e.g., if gate is detected via accelerometer don't activate collection by other sensors).

The accelerometer can be monitored to determine (by looking at amplitude, frequency) whether the user is walking/running. If so, this data can be used to determine energy expenditure (intensity of acceleration, number of steps, duration of activity). If there is no gross motion, the surface deformation signal is analyzed to see if the jaw is moving. If the jaw is moving the algorithm determines if the person is speaking by running speech detection algorithms on the acoustic signal derived from the bone conduction microphone.

Signal from the microphone can be processed to see if the person is talking by using a speech detection algorithms. A speech detection algorithm could be implemented by segmenting the signal into fixed size frames of 10 ms and extracting spectral features for each segment. [Furui “Cepstral Analysis Technique for Automatic Speaker Verification,” in IEEE Trans. Acoust., Speech, Signal Processing, vol. ASSP-29, pp. 254-272, April 1981]. Speech could be detected at the frame level by using a Gaussian Mixture Model (GMM) [Duda et al. “Pattern Classification Second Edition,” John Wiley & Sons, Inc., 2001]. A state machine could be used to make a final decision by tracking how many consecutive frames were detected as speech. Detected speech can be processed using hidden Markov models (HMMs) [Young et al. “The HTK Book for Version 3.2,” Cambridge University Engineering Department, Cambridge University, 2002] or other speech recognition algorithms. Information extracted from the speech signal could be used to control the device, for example to report the type of food to be ingested or to record a short message.

If a user is not walking or talking, the microphone and deformation sensors signals are used to create an Event Log (Event log box, left bottom side of FIG. 1) which includes the time, the digital signal itself (when sufficient memory is available), and additional information extracted from the signal such as energy, spectral features, and chew information. The information is stored into a database in the memory of the device. Such an Event log is stored by a memory unit (e.g. NAND flash) of the device and used to determine eating habits of the user. The stored information is also used to build an offline database for further analyze user eating habits and to refine the statistical models used to extract eating information from the digital signal.

For example, the Event log can include individual chews whose timing sequence be used to infer swallows (break in chewing patterns) using an energy detection algorithm. Information from several measurements such as the duration, frequency, energy, spectral distribution and number of chews can be combined to predict swallowed bolus volume (further detail is provided in the Examples section).

HMM algorithms can be used to process snippets of sound and pressure waves from the microphone and deformation sensors to determine where the chews occur in the signal and also to classify the type of ingested food from which refinement of the food volume estimation can be made and caloric input can be estimated (using a look up table stored by the device).

The unique acoustic and deformation patterns for drinking or eating soft food (e.g. ice cream) can be derived from the sensors and processed into the event log.

Periodic assessment of the sensors can be used to determine if the user is wearing the device or not in which case these sensors will generate different signals (see the “compliance and usage monitoring” box right side of FIG. 1).

The Event Log can be analyzed by various expert system modules to fashion an appropriate feedback to the user. One or several such algorithms (dynamic baseline control and virtual Lap Band boxes, right side of FIG. 1) can be run in parallel and the feedback from each can be placed into the Feedback Processing (Feedback processing box, right side of FIG. 1) module which in turns sequences the appropriate feedback to the user through a sound transducer (circle, lower right corner of FIG. 1).

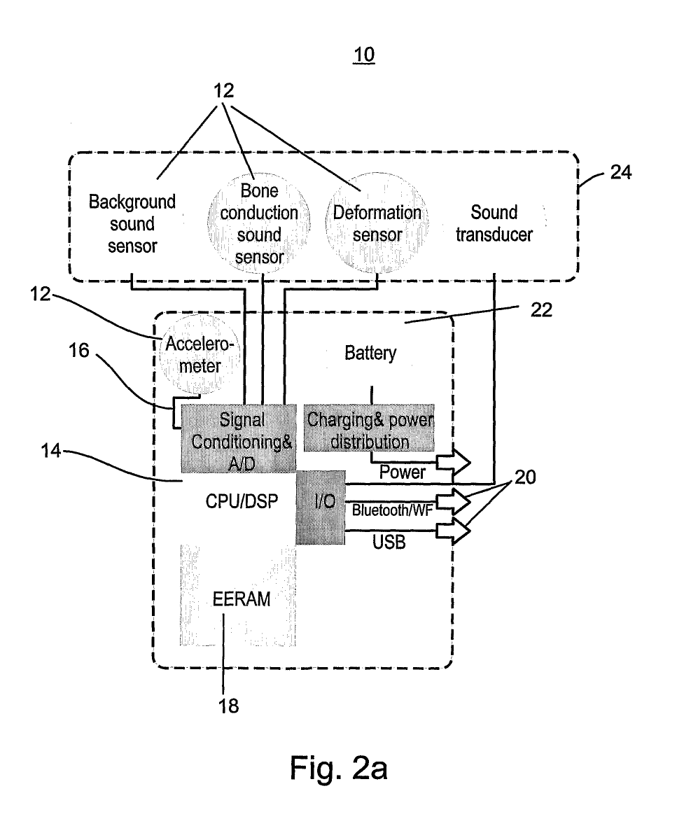

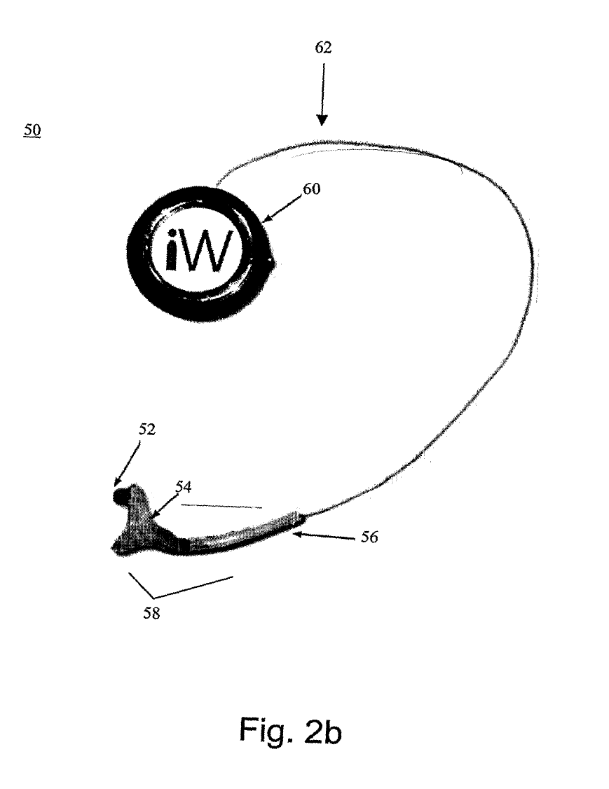

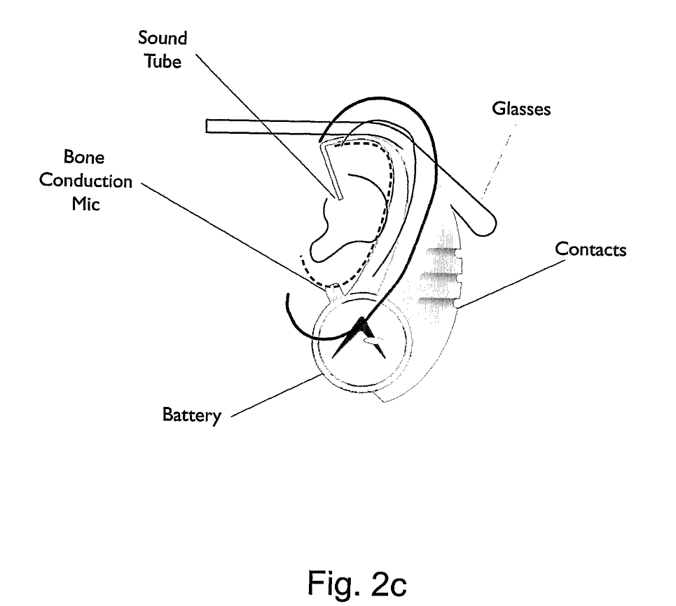

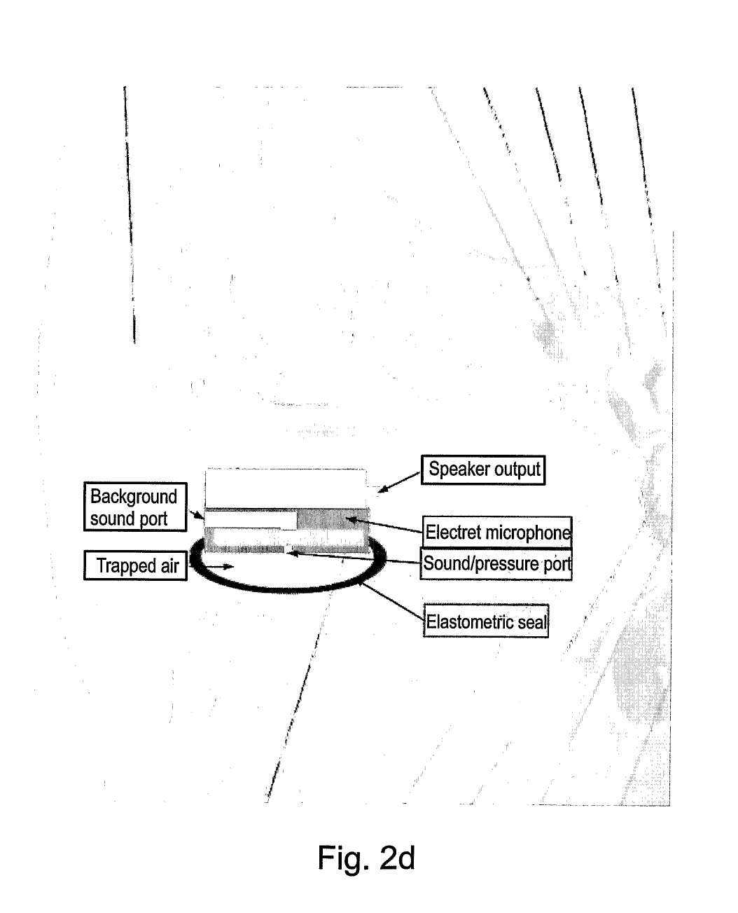

FIG. 2a is a box diagram illustrating the components of the present device which is referred to herein as device 10. Device 10 includes a low power micro processor 14 (e.g. DSP) which processes a signal from a signal conditioning unit 16. Signal conditioning unit 16 obtains signals from one or more sensors 12 which perform the function of a bone conduction microphone, a background sound sensor, a deformation sensor and an accelerometer. Processor 14 extracts the information from the signal conditioned by unit 16, performs the analysis and generates the appropriate feedback when needed. The software and voice feedback files are stored in memory 18 (e.g. EERAM) on board. Both can be updated through one or more external communication ports 20. Processor 14 also performs power management and external communications. Preferably, all of these components are combined into one application specific integrated circuit (ASIC) which is housed along with sensors in a single device housing. A rechargeable or disposable battery 22 can power processor 14 and all its communication ports. Device 10 can dock to a docking station which includes a communications port (USB, wireless etc) which can be used to retrieve software (e.g. algorithm updates) and feedback modules. Processor 14 can also communicate with other devices such as a cell phone, cordless phone, PC via standard short range RF link such as WiFi or Bluetooth. Device 10 further includes a speaker 24 (sound transducer) for relaying feedback and providing coaching to the user.

Sensor array 12 records the sounds/deformation made by chewing, swallowing, biting, sipping, and drinking as well as body movement in the manner described above. Features related to eating and physical activity are extracted and classified as events (e.g. chew log) as described above.

FIG. 2d illustrates in-ear placement of the sound and canal-deformation sensors of the present device, a combined in-ear—behind the ear configuration is illustrated in FIG. 2c, in such a configuration the power supply and processor are housed in a device body which is configured for behind the ear positioning (without interfering with an ear piece of eyeglasses), a bone conduction microphone is positioned behind the ear and against the bone and s speaker (sound tube) is positioned at or in the opening of the ear canal. A modified in-ear configuration which includes an attached (neck-mounted) accelerometer is illustrated by FIG. 2b which is further described in the Examples section below.

The basic log of activities generated by the detection algorithms can be combined with an expert system of rules, user behavior history and weight loss objectives to generate appropriate feedback and track user compliance.

For example, a ‘dynamic baseline’ algorithm can utilize the chew log and related parameters as a proxy for caloric input. By inputting user weight periodically, the algorithm automatically adjusts to their base line diet.

A ‘virtual lap band’ algorithm (VLB) can utilize mastication energy (as determined from the deformation sensor), frequency and duration time and the user's behavior profile (as stored by the device) as a proxy for estimating ingested rate of food volume. Using input from such an algorithm, the present device can then mathematically simulate the physiological behavior of a Lap Band bariatric implant in order to generate user feedback (stop eating for a while, continue eating, pause, drink, etc.) which allows the user to experience natural satiety and reduce food intake.

Similarly, the device can utilize an aroma emitting device (e.g. nostril mounted tube connected to a source of aromatic compound) and release specific scents when a predetermined eating pattern is detected. For example, if a user exceeds a predetermined number of chews or chew frequency, the device of the present invention can release an appetite curbing aroma [Yeomans Physiol Behav. 2006 Aug. 30; 89(1):10-4. Epub 2006].

It has been documented that in some cases, people continue eating after the first two bites for the mere sensual pleasure. The present device can use the acoustic fingerprint of the food consumed and determine when a user eats more then two bites of the same food to alert them to stop.

The above described algorithms can be modified according to user needs and periodic user examination by a physician.

The device can adapt to changing eating habits of individual users throughout the day or over the course of weeks or months by learning the specific food preferences of the user and “closing the loop” using one or more of the body mass detection means described above. If the user changes their food preference over time, towards higher energy density foods for example, the system can recognize that fewer swallows are required to reach the caloric intake budget because the weight of the individual will be higher for a given number of swallows. Or, as another example, if the person is very physically active, the system can increase the caloric budget to maintain the desired energy balance.

The present device can gather information about the user's weight by communicating through a wired or wireless connection with a body mass measuring system (such as a traditional weighing scale), a body fat measuring system (such as a body fat composition scale, hand-held fat detection system, or calipers), a specially designed garment or other physical measurement of one or more body parts; or a continuous body weight measuring means, such as load sensors placed in the shoes of the user or the seat of the user's vehicle.

The device can be adapted to detect patterns in body mass or fat content measurements by one of the means described above and use such patterns to calibrate, with or without user input, the allowed caloric input based on desired weight targets and time periods or to adjust the detection algorithms.

The device of the present invention will also incorporate features which enable social interaction between users of the device (via, for example, the web), enabling voice feedback coaching based on the behavior algorithms and the like.

User compliance can also be measured by the present device. The device can measure usage and monitor compliance of voice commands issued to the user. For example, when asking the user to wait for a period of time before resuming meal, the device can monitor if the user stopped or continued eating. By monitoring background sounds the present device can determine whether a human is wearing it or not.

The data generated by the system for each subject can be integrated and/or aggregated into a flat or relational database containing the behavior related activity signatures collected from a plurality of subjects over a time period. Such a database can contain proprietary data, publicly available data, anonymously collected data, and/or data collected with subject identification information. Data could be collected at a variety of levels, from raw recordings of sensor data, processed sensor data, activity-related signatures, or high-level behavioral data. Such a database would be useful for establishing norms, averages, trends, classifications, calibrations, historical behaviors, reference sets, training data, statistical tests, clinical trials, targeted marketing, third party interventions, and relative scores in a stand alone manner as pure data or as an integral part of the system used by individual subjects. Such a database can be cross referenced to other databases. By way of example, a database of the physical activity patterns or ingestion patterns of many subjects can be cross referenced to a database of their health, medical, exercise, drug use and/or weight records. Unexpected relationships are likely to emerge from an analysis of such a database. For example, a drug company doing a clinical trial of a drug may find it useful to measure the ingestion behavior of the patients in the trial to detect any changes of eating or drinking behavior of the patients either as a result of taking the drug or as a side effect. By way of a second example, an aggregated database of the ingestion related motion or acoustic energy patterns detected for a plurality of users can be used to train the algorithms used to convert these patterns into classifications of ingestion behaviors. Additionally, such a database can be used to let a subject know where he or she is performing relative to other users of the system.

The database can correlate the effect of various algorithms on the eating behavior, compliance and target weight accomplishments of users. This will enable to test the efficacy of various eating modification algorithms and determine their applicability to sub groups within the database eventually enabling matching user profiles (as determined by a questionnaire and/or physical examination) with specific algorithms.

The device can be pre-programmed at the factory, at the point of sale or at home to require no user intervention other than wearing the system in a pre-set weight reduction or weight maintenance mode. The device can communicate directly with the internet, a personal computer, laptop computer, pocket computer, personal digital assistant, cell phone, pager, wristwatch, a dedicated control panel on the system or a dedicated external system for programming the system and/or the physical activity monitor. The user can program the device and set preferences via voice commands.

The device can be set by the user to issue occasional summary reports or real-time messages or stimuli before, during or after a meal. The messages or stimuli can be set by the user or a third party to be anything from gentle reminders to un-ignorable in terms of their intensity. The device can be programmed by the user or a third party to not be controllable by the user for a set period of time or until certain events or parameters occur. This could include not having the user be able to turn off the machine at all, or more than a set number of times per day.

The device will be able to handle exceptions and deal with them intelligently. For example, if the user did not wear or turn on the system for a set period of time, the device can beep or otherwise alert the user until the system is put on by the user. Since the system will be able to keep track of time and a usage history, it can use average caloric consumption values from previous usage data for the skipped measurements. The device will detect a skipped meal by not detecting swallows at the appropriate time and then carry forward the unused caloric budget to the next meal. The device can detect an increase in physical activity and increase the caloric budget for that same day.

The device of the present invention preferably can be configured to communicate directly with the internet, a personal computer, laptop computer, pocket computer, personal digital assistant, cell phone, pager, watch, a dedicated control panel on the system, or a dedicated external system in order to provide audio or visual feedback, summary statistics and trends, the unprocessed or processed data collected by the chew or swallow sensors and/or the caloric expenditure monitor.

The device can produce synthesized or prerecorded speech messages to the user through a speaker, an in-the-ear speaker, or through bone conduction technology as described above. It can assemble speech snippets from a pre recorded celebrity or actor to give the user the sensation of a personal coach.

A bone conduction speaker or a miniature sound transducer near the ear canal will not generate significant sound to the outside world, and therefore the microphone component for picking up the user's voice, internal cranial sounds and/or ambient sounds in the user's environment usually will not need to cancel out the feedback of the speaker sounds. This should enable full duplex communication with a minimum of signal processing for applications such as cell phone headsets for example, even in the case where the speaker and microphone are co-located or in close physical proximity. In another embodiment, time-division duplex techniques can be used to separate the microphone input from the speaker output.

Software and electronics can drive a transducer in either microphone or speaker mode. The software and electronics could also deconvolute the signal to allow simultaneous use of the transducer in both microphone and speaker modes.

The device can produce a visual display of information for the user, using for example lights, symbols, graphics or numbers. The device can also vibrate a coded message to the user.

The device can be part of a formal medical or diet plan where the device would collect data and transmit them in batch mode or in real time to a server or other data sharing mechanisms where a clinical specialist, doctor, nurse, dietician, friend, family member, other device users, or any other relevant third party can be alerted for possible intervention.

The device can be used to qualify or classify candidates for pharmaceutical or surgical procedures by monitoring eating patterns beforehand. For example, before qualifying for insurance reimbursement or a referral for bariatric surgery, a patient would submit the eating patterns as collected by this device to their caregiver for analysis and the risk and likely outcomes of the surgery can be simulated, analyzed, and predicted.

The device can provide positive feedback when the user stops eating at or below the caloric budget threshold using any of the means above.

The device can alert the user when not enough physical activity has been detected, thereby providing an incentive to exercise in the proper amount. Conversely, the device can provide positive feedback when enough physical activity has been detected.

The device can provide ongoing feedback as to where the person is relative to the caloric budget before during or after each meal or other period of time such as a day or week.

The device can proactively predict low blood sugar situations by detecting the time between meals relative to historical eating and activity patterns and alert the user to either eat or provide a preventive message to avoid binge eating that may be triggered by low blood sugar situations. Conversely, the device may proactively predict high blood sugar situations and warn the user to stop eating or take his or her insulin medication.

The device can issue “warning signals’ prior to alerting the user with full force to allow for gradual cessation of eating. The device can generate appetite generating messages or sounds in order to encourage a person to eat or appetite suppressing messages or sounds in order to encourage a person to stop eating.

The device can detect eating rate and warn the user to slow down, allowing for natural satiation signals to build up and thereby allowing people to eat less.

The device can encourage the user to take smaller bites by detecting chewing time and intensity. Smaller bites will cause the user to eat slower and therefore eat less.

The device can issue “pre-emptive” signals or voice messages with psychological content, either positive or negative, before or during the meal to remind and encourage the person to eat less.

The device can also ‘remind’ the user to savor the food ingested by, for example, promoting awareness of tastes and smells associated with the food ingested. For example, the device can instruct the user to slowly experience the tastes on your tongue, stop eating and savor, thereby inducing ‘mindful’ eating. It is proposed that such mindful eating will decrease the caloric intake of an individual.

The device can act as a virtual personal eating and physical training coach, providing real time feedback and encouragement. Attention is given to the psychological aspects of the disorder being treated by this device. For example, after detecting a binge eating episode, the device will surmise that the user may be feeling disgusted, guilty and depressed by his or her actions, and the device will therefore issue encouraging and supportive remarks to ameliorate these feelings, much like a real psychological coach would do.

The device can be calibrated by simply being worn by the user for a period of time and then set manually or automatically to preset “diet modes” based on alerting the user to stop eating when the device senses the consumption of a preset or user-definable percentage of the food consumed during the calibration period. For example, after being worn by the user for a week or so, the device will have learned the eating habits of the user. The device then goes into “diet mode” automatically and alerts the user when he or she has eaten say 90% of each meal, thereby eliminating the last 10% of the total calories otherwise consumed by the user over the course of a day as determined by retrospective eating behavior. Relative to skipping meals altogether, to not eat the last 10% of the calories towards the end of a normal meal is easier, because some satiety has already set in. At some point the user becomes conditioned to this amount of food and may not even trigger the warning message, in which case the user can receive positive feedback from the device. Once the target weight is achieved, the device can go on “weight maintenance mode” which may require a reduction of only 0.1%-10% of the calories typically consumed by the user who would otherwise creep back up to a higher weight without the device.

The device can interact with the user and ask the user questions in real time, such as to inquire about the user's eating and activity plans for the day, and only then determine the right course of action. For example, the device can detect overeating and ask the user if he or she intends to perform physical activity later that day. If so, the device increases the calorie budget for the meal, and reminds the user later to work out. Furthermore, the device can alert the user how much exercise will be required to work off the amount of food being ingested, partially as a deterrent to prevent the user from over-eating. This form of an interactive “relationship” between the user and the device will increase the user's emotional connection and desire to use the device.

The user can communicate with the device through voice commands or by using teeth clicks or other non-verbal sounds as responses to queries from the device. Using such sounds substantially reduces the computational and power burden on the device.

Thus, the present invention provides a device which can be used to monitor and modify eating behavior of a subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computing platform using any suitable operating system. In any case, selected steps of the method and system of the invention could be described as being performed by a data processor, such as a computing platform for executing a plurality of instructions.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### Device Mounting Considerations and Experience

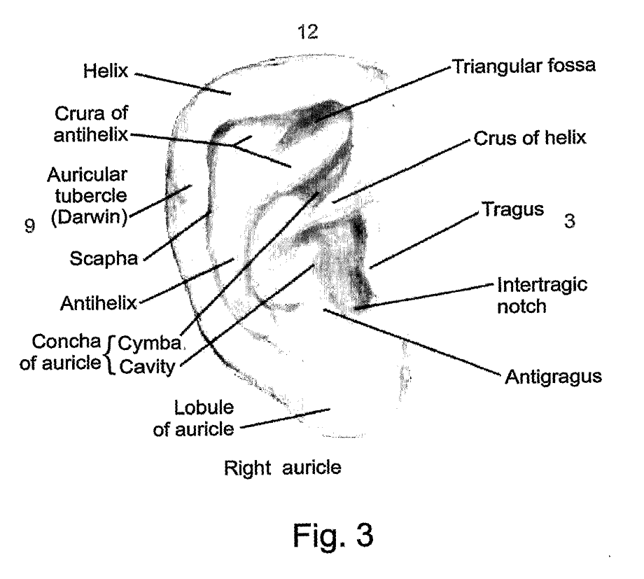

Since gravity can be a factor in device operability the microphone should press against the skull slightly above where the skull connects to the helix of the ear (See FIG. 3). The top of the ear at the 11 o'clock position (12 o'clock is at the top of FIG. 3) is most likely the optimal location since the device is most stable at that location, the jaw muscles are still pronounced enough and there is motion, the wedge formed by the helix and skull naturally keeps the microphone in contact with the skull and gravity will help fix the device in this location (bottom spring arm helps). This positioning should take into account eyeglasses which might interfere with placement and that jaw muscles are not as pronounced as they are right under the ear.

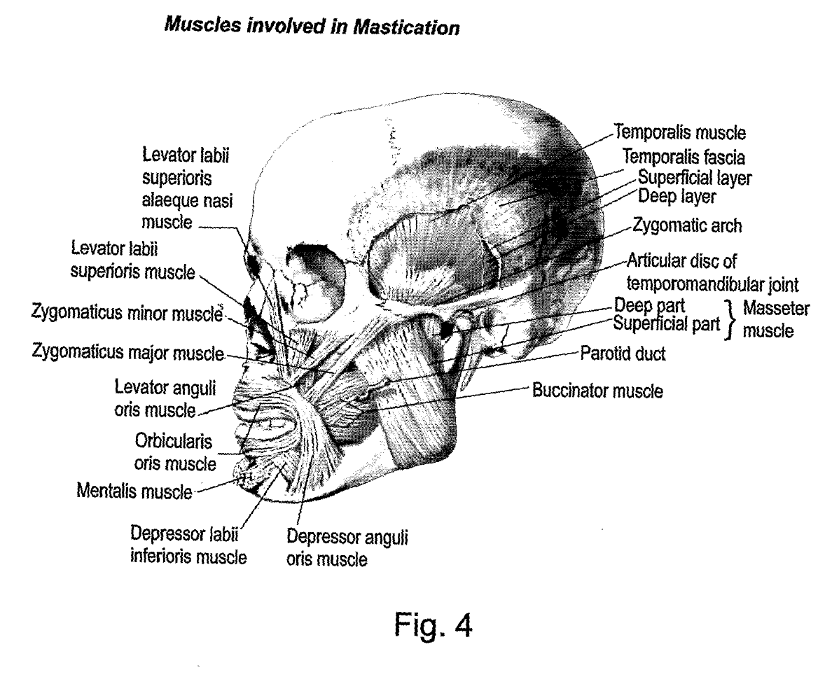

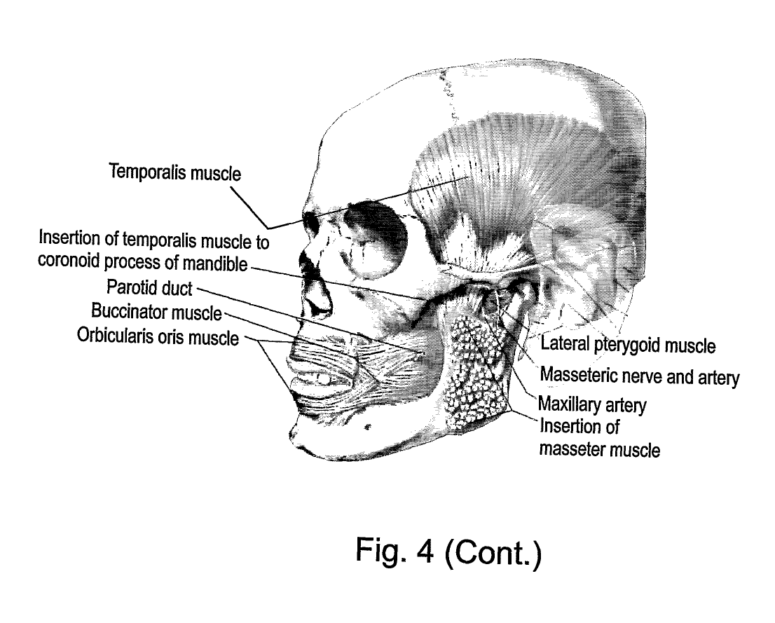

The top image of FIG. 4 illustrates insertion of the Temporalis muscle into the skull bone. The lower image of this Figure illustrates an ear superimposed on top of the skull and highlights (in green) a region where motion caused by muscular contracture due to chewing can be detected.

FIG. 2b illustrates one specific design of the present device which takes into consideration the device mounting parameters discussed herein. The device (which is designated as device 50 in this Figure) includes a bone conduction microphone 52 and speaker 54 which are designed to be positioned within an ear without occluding the ear canal. Device 50 further includes a retention clip 56 for securing an ear portion 58 of device 50 which includes microphone 52 and speaker 54 to an ear of the user (see inset image of FIG. 2b). If the microphone used has a low frequency dynamic range, the deformation of the ear canal surface by activation of the musculature moving the jaw results in deformation of the spring loaded elastomeric seal which in turn creates pressure changes which can be detected with the microphone as low frequency voltage changes (e.g. an Electret microphone). Hence one detector can pick up both acoustic and deformation signals. Alternatively, the elastomeric material can be lined with a piezo electric film which generates a voltage when deformed by the jaw movements as described above. Device 50 further includes a neck mounted portion 60 which includes a CPU, an accelerometer and a battery. Neck portion 60 is connected to ear portion 58 via wire 62.

### Example 2

### Monitoring of Daily Eating Times in Human Subjects Using the Device of the Present Invention with Feedback

A long-term study of the device was conducted in the field by tracking a 21 year old male subject (weight 214 lbs, height 70 in, BMI-30,8) for 22 weeks.

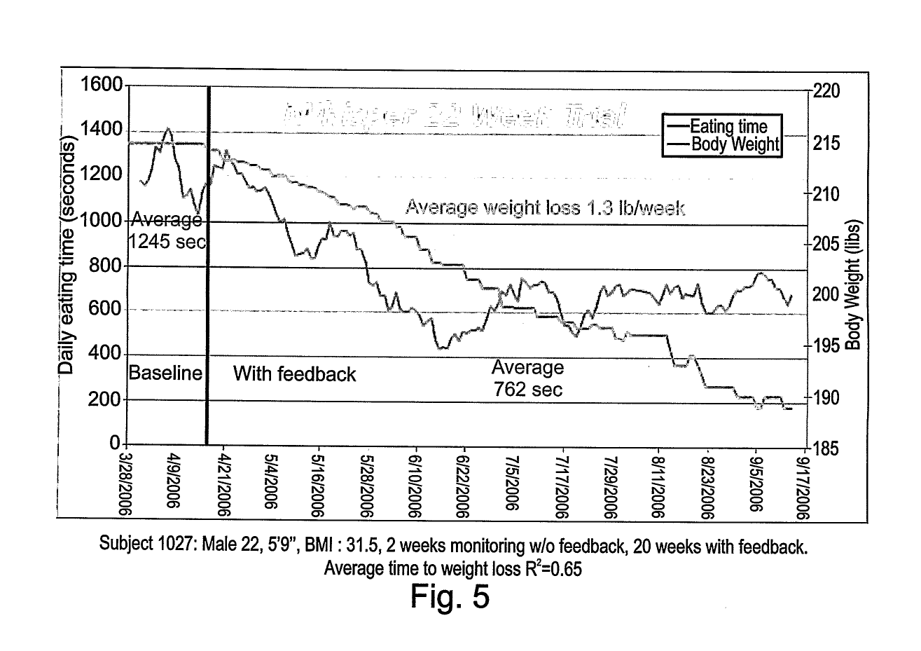

The goal of the study was to observe the relationship between eating duration and weight. The subject was instructed to wear iWhisper for every meal. A simple weight loss program that required him to limit his eating duration on a daily basis was implemented. The device presented the eating duration information in two ways: (1) on the PDA screen using various daily statistics, and (2) using simple spoken feedback “You are approaching 200 seconds.”. The subject was instructed not to exceed a target daily eating duration. The hypothesis was that continuous real-time feedback would help the subject plan and control food intake. The initial target duration was set at 1000 seconds based on the average first week of usage and was reduced further over time in increments of about 10% until the weight loss rate stabilized at about 1 Lb/Week. The long-term target was set at 800 seconds. It took the subject a few weeks to fully adjust to the new regimen. After this initial period, his discomfort diminished and he successfully used the device for losing weight with little support from the experimenter. The subject continued using the device beyond the initial 6-week target. The device was synchronized with a server on a daily basis. Audio and recognizer output data was downloaded to the server while software updates were uploaded to the device. To improve eating recognition accuracy, the statistical models were retrained using audio recordings downloaded from the device. A chart showing weight and eating duration over time is shown in FIG. 5. The chart clearly shows a dramatic drop in eating duration after a couple of weeks. The peak observed at 1400 seconds is consistent with his reporting of uncontrolled eating. Once eating duration stabilized in the 600-800 second range, weight loss rate stabilized at an average of 1 Lb/week.

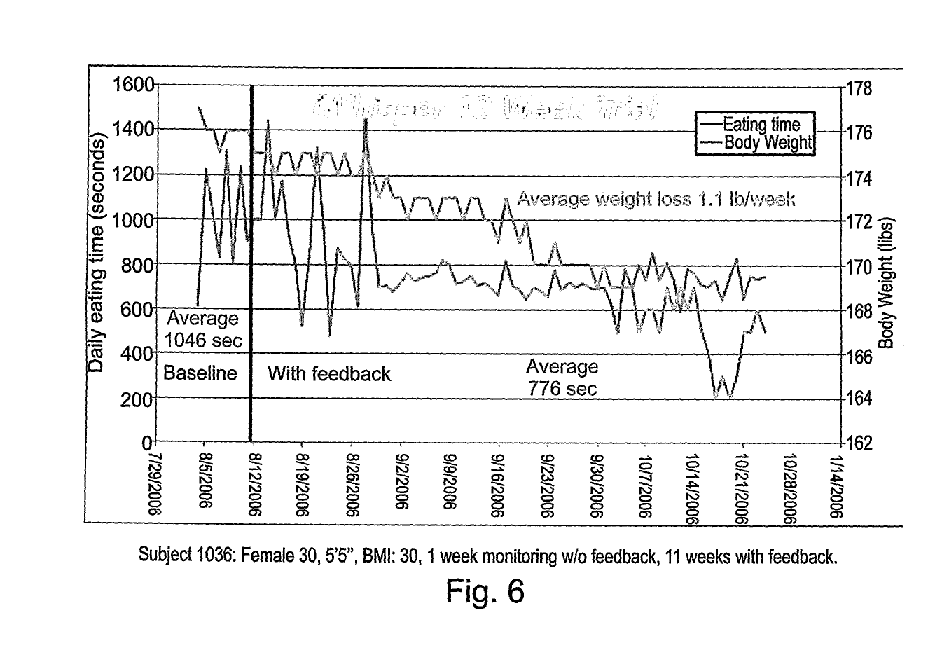

A medium-term study of the device was conducted in the field by tracking a 30 year old female subject (weight 177 lbs, height 65 in, BMI-30) for 12 weeks (see FIG. 6).

The goal of the study was to observe the relationship between eating duration and weight, to establish the comfort of the ear sensor and the acceptance of the voice feedback. The subject was instructed to wear iWhisper for every meal. A simple weight loss program that required the subject to limit her eating duration on a daily basis was implemented similar to that described in Example 1. The device presented the eating duration information in two ways: (1) on the PDA screen using various daily statistics, and (2) using simple spoken feedback “You are approaching 200 seconds.”. The subject was monitored for 2 weeks where the baseline eating time of an average of 1046 seconds per day was measured. The subject displayed a large day to day variability which is common in subjects trying to lose weight (‘least or famine’). Subsequently the feedback was turned on and the subject was asked to keep her total daily eating time to less than 800 seconds. Within a couple of weeks the subject achieved an average of 776 seconds per day and lowered the day to day variability significantly. This demonstrates another positive attribute of the real time accurate measurement with feedback which allows the user to gauge their progress throughout the day and ‘budget’ their eating time appropriately and thus avoid mindless eating that leads to over consumption of food and increased caloric intake. The subject lost about 1.11b/week throughout the 10 weeks of active feedback.

### Example 3

### Sound Processing

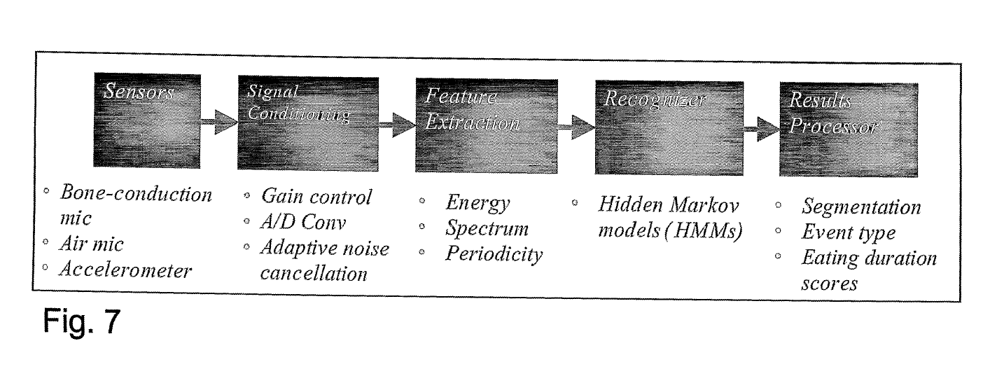

FIG. 7 illustrates sound processing as conducted by the present device. An array of sensors including a bone-conduction microphone, and air microphone located near the bone-conduction microphone and an accelerometer mounted on the ear send analog signals to analog-to-digital converters. Unlike a conventional air microphone, the bone conduction microphone only captures the sounds transmitted through the bones and muscles, rejecting ambient noise, providing a very desirable signal-to-ambient-noise ratio [Zhang 2004 Proc. International Conference on Acoustics, Speech, and Signal Processing, Montreal, Canada, 2004]. The ear canal provides good access to sounds originated in the mouth and to subsonic signals generated by the movement of the jaw. The sensor is a miniature electret microphone with an integrated amplifier (Sonion 9723 GX) which is typically used in hearing instruments. A comfortable earpiece that holds the microphone inside the ear canal without obstructing it was designed and employed. The microphone is covered with a rubber bubble that makes contact with the skin. In addition to the microphone, the earpiece also includes a miniature speaker that is used to provide spoken feedback to the user. The following describes an implementation that only uses the bone-conduction microphone. However, additional features could be extracted from other sensors to improve the event classification accuracy.

The signal sent by the bone-conduction microphone captures vibrations from multiple sources including jaw muscles, jaw bones, teeth, fluids in the mouth, compression of the food, breathing, vocal folds, etc. The signal is also affected by various factors including the size and shape of the subject, the eating style, e.g. chew force, chew rate, number of deglutitions per bite, duration of intra-meal pauses. Because of the high variability of the signal, statistical pattern recognition techniques [Duda et al. “Pattern Classification Second Edition,” John Wiley & Sons, Inc., 2001] are used to model the eating microstructure and automatically segment the signal into various events.

The signal sent by the bone-conduction microphone is sampled at 8 Khz with a precision of 16 bits. Adaptive noise cancellation may be used to remove background noise by subtracting a derivative of signal captured by the air microphone from that of the bone conduction microphone [Widrow et al. “Adaptive Noise Cancelling: Principles and Applications”, Proceedings of the IEEE, vol. 63, pp. 1692-1716, Dec. 1975]. The noise cancellation stage could be implemented prior to the feature extraction stage. The digital signal from the bone-conduction microphone is parameterized using fixed size frames of 200 ms and an analysis window of 500 ms. For each frame a 27-dimensional feature vector that includes energy and cepstrum and the corresponding first- and second-order differences is computed [Furui “Cepstral Analysis Technique for Automatic Speaker Verification,” in IEEE Trans. Acoust., Speech, Signal Processing, vol. ASSP-29, pp. 254-272, April 1981]. Features are designed and tuned to maximized discrimination between target classes, for example eating versus non-eating events. A large number of features could be computed and combined into a single feature vector of lower dimensionality than the original dimension of all the input features by using linear discriminant analysis (LDA) [Duda 2001 Ibid]. The recognizer is a statistical classifier capable of classifying frames into predefined classes such as eating, drinking speaking, silence, walking, etc. One method for segmenting a digital signal into time-varying source classes is to use continuous-density hidden Markov models (HMMs) which has successfully been used in many speech classification applications including speech recognition, speaker classification, language recognition, and so on. The output of the recognizer is used by the Results Processor module to generate the final response that is used by the present device to provide feedback to the user. For example, average chewing duration which is highly correlated with caloric input.

For each a continuous-density hidden-Markov model was trained using the HTK toolkit [Young et al. “The HTK Book for Version 3.2,” Cambridge University Engineering Department, Cambridge University, 2002]. Optimal performance was achieved using 2 states and 20 Gaussian mixture components for each model. Each model corresponds to one of the target events. To estimate the optimal recognizer parameters, a large database of labeled sound examples is utilized. The accuracy of the recognizer depends on the quality and richness of the sound database. Once the HMM parameters are estimated, the next step is to search for the most likely state sequence given the observed data and the model. The optimal state sequence is efficiently computed using the Viterbi search algorithm [Kruskal “Time Warps, String Edits, and Macromolecules: The Theory and Practice of Sequence Comparison,” Reading, Mass., Addison-Wesley Publishing Co., 1983]. The Viterbi search outputs the optimal state sequence and time alignment information. The state sequence is used to hypothesize the sequence of events. The HMM states could be designed to model long-term events such as “eating” or more detailed events such as the microstructure of eating, for example micro-events such as “bites”, “chew sub-components”, and “swallows”. Because not all event sequences are equally likely, the model is defined by using a “stochastic language model” [Jelinek “Statistical Methods for Speech Recognition. Cambridge,” MA, MIT Press, 1998] to constrain event transitions using prior knowledge about the sequence of events observed in the training set.

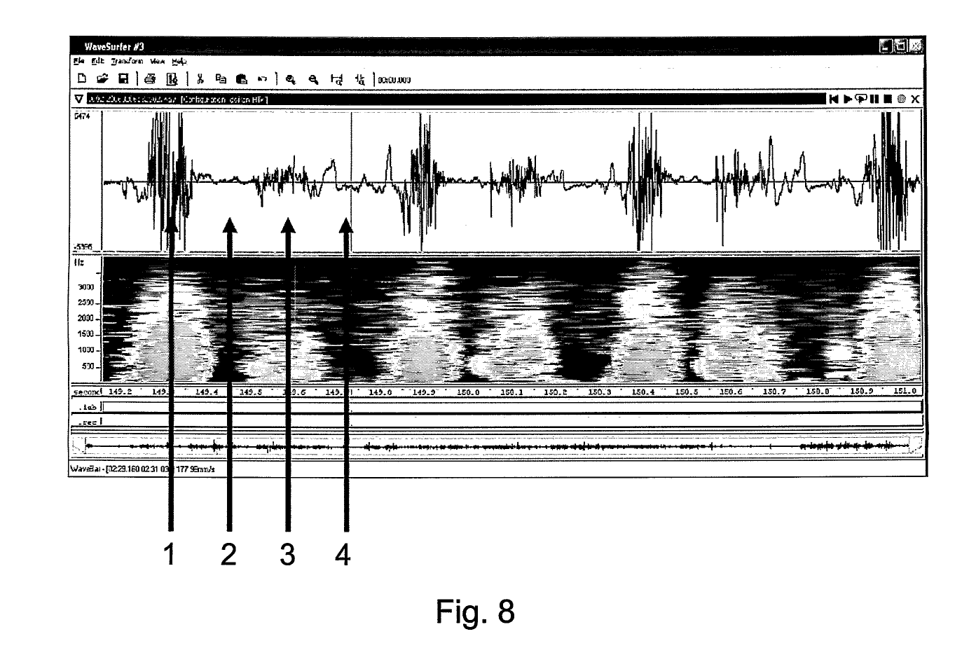

A sample eating sequence is shown in FIG. 8. A chew cycle is characterized by four distinct phases. First the jaw closes and fractures the food which emits acoustic vibrations. In the second phase the jaw is closed producing little signal activity. In the third phase the jaw opens emitting significant acoustic energy but with less intensity than in phase 1. The signal in phase three is generated as the movement of the jaw and the tongue releases and repositions the food in preparation for the next chew cycle. Phase four is the quite phase that completes the cycle. HMMs could be used to model the sub-chewing events.

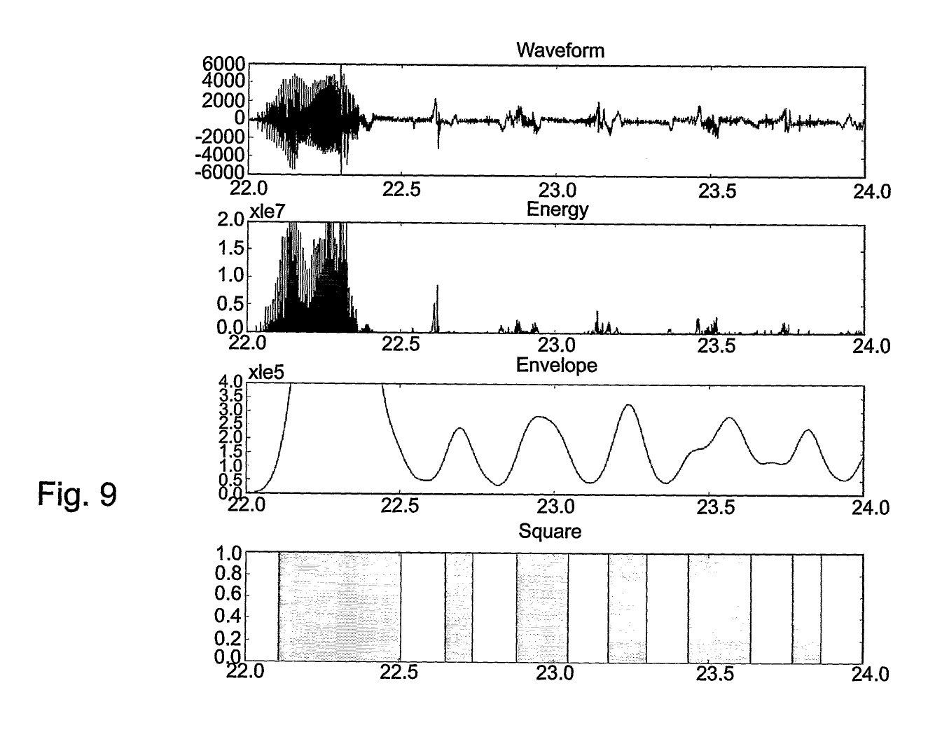

FIG. 9 illustrates a typical signal that has a speech segment followed by two chews. A simple approach for segmenting the signal into chews is to rectify the signal, pass it through a low pass filter and segment using a threshold. This technique could be used to obtain a clean very low frequency signal that correlates with the movement of the chews when the subject is eating. A possible feature could be extracted from this signal and be used in combination with normalized energy and spectrum.

A database of sounds was recorded in the lab and hand-labeled by a panel of human listeners. A partition of the database was used to train the HMMs. A separate partition was used to evaluate the accuracy of the system. To record the data in the field a portable data collection tool application for Windows Mobile was designed and implemented on a Dell Axim PDA running Windows Mobile 2003 which provides internal analog-to-digital conversion for audio signals. The sensor described above was used to collect audio and the recognition task was set to classify the audio recordings into eating and non-eating segments. The accuracy of the classifier was evaluated by computing the false negative rate (eating segments classified as non-eating segments) and false positive rate (non-eating segments classified as eating segments). The HMM models were trained for the following events: Silence, Speak, Drink, and Eat using the training data set. Noise segments were merged with Silence segments.

Table 1 below shows the results for various algorithms. A significant performance improvement was achieved by adjusting algorithm parameters, such as the length of the analysis window, spectral warping, filterbank design, and the size of feature vectors. A bigram statistical language model [Jelinek “Statistical Methods for Speech Recognition. Cambridge,” MA, MIT Press, 1998] was created using the training set. A bigram is used to determine the probability of an event in a sequence given that the previous event is known. The bigram probabilities are computed from a training data set. To estimate the probabilities an independence assumption that each event only depends on the previous event is made. This method can be generalized to using n-grams where the probability of an event is predicted from the sequence of the previous n events. To maximize the use of the training the total number of parameters in the models was increased by varying the number of HMM states and the number of components in the Gaussian mixture distribution. Further improvements to performance were achieved by equalizing the acoustic channel.

TABLE 1
algorithms

| | False Negative | False Positive |
|---|---|---|
| Experiment | [%] | [%] |
| Baseline | 45.0 | 4.9 |
| (No tuning, no language model). | | |
| Tuned signal processing parameters. | 34.4 | 4.8 |
| Added bigram event language model. | 22.1 | 7.7 |
| Increased number of model parameters. | 18.5 | 9.2 |
| Channel equalization using cepstral | 6.3 | 8.6 |
| mean subtraction. | | |

TABLE 2
Results by Subjects, channel equalization condition.

| | | | | True | False |
|---|---|---|---|---|---|
| | | | | Positive | Positive |
| Subj. ID | Gender | Age | Food | Rate [%] | Rate [%] |
| 1003 | F | 21 | Beef, pasta, fruit | 1.8 | 16.1 |
| 1006 | F | 48 | Sandwich, | 2.5 | 10.9 |
| | | | apple, carrots | | |
| 1011 | F | 71 | Sandwich, | 9.9 | 5.0 |
| | | | grapes, cookie | | |
| 1016 | F | 45 | Fruit, nuts | 8.0 | 2.7 |
| 1018 | F | 33 | Sushi | 9.4 | 1.0 |
| 0001 | M | 27 | Sushi | 14.6 | 0.5 |
| 1001 | M | 48 | Sandwich, | 3.9 | 0.0 |
| | | | barley salad | | |
| 1002 | M | 77 | Sandwich | 3.3 | 7.5 |
| 1017 | M | 20 | Sandwich | 7.2 | 4.1 |
| 1022 | M | 45 | Sandwich, chips | 2.4 | 38.6 |

| | | |
|---|---|---|
| AVERAGE | 6.3 | 8.6 |

A prototype of the present device was tested in the field, the results are shown in Table 2. The hardware was a Dell Axim PDA and the sensor was the custom-designed bone conduction microphone described above. Various aspects of the device including the user-interface, visual and spoken feedback approaches, and weight control strategies were evaluated.

TABLE 3
composition of the database

| Subj. | | Height | Weight | | | Audio | Annotation | Annotation |
|---|---|---|---|---|---|---|---|---|
| ID | Gender | [in] | [Lbs] | BMI | Age | [hrs] | [hrs] | [%] |
| 1027 | M | 69.0 | 214.4 | 31.7 | 22 | 171 | 121 | 71% |
| 0002 | M | 71.3 | 170.0 | 23.5 | 40 | 81 | 68 | 84% |
| 1024 | M | 71.1 | 164.0 | 22.8 | 31 | 66 | 30 | 46% |
| 1031 | F | 65.0 | 204.0 | 33.9 | 42 | 54 | 50 | 92% |
| 1034 | F | 69.0 | 247.0 | 36.5 | 41 | 44 | 36 | 82% |
| 1038 | F | 64.0 | 177.0 | 30.4 | 35 | 40 | 32 | 81% |
| 1036 | F | 65.0 | 177.0 | 29.5 | 30 | 27 | 23 | 83% |
| 1028 | F | 68.1 | 170.0 | 25.8 | 39 | 23 | 22 | 98% |
| 1037 | F | 63.0 | 142.0 | 25.2 | 58 | 22 | 15 | 68% |
| 1030 | F | 64.0 | 163.0 | 28.0 | 29 | 12 | 12 | 100% |
| 1001 | M | 71.1 | 175.0 | 24.4 | 45 | 8 | 7 | 85% |
| 1039 | F | 63.0 | 132.5 | 23.5 | 27 | 8 | 2 | 29% |
| 1026 | F | 66.9 | 128.0 | 20.1 | 29 | 7 | 7 | 100% |
| 1033 | F | 67.0 | 189.0 | 29.6 | 30 | 6 | 6 | 100% |
| 1035 | F | 70.0 | 306.0 | 43.9 | 32 | 3 | 0 | 0% |
| TOTAL | | | | | | 572 | 432 | |
| AVERAGE | | 67.2 | 183.9 | 28.6 | 35 | 38 | 29 | |

The device was used to record 572 hours of data from 15 users (Table 3). Of those recordings, 432 hours have been annotated by human listeners using a custom-made annotation tool (Table 4). The human listeners segmented each recording into a disjoint sequence of events (eating, drinking, speaking, walking, silence, noise, and other).

TABLE 4
the amount of data (in hours) for all subjects by class

| | EAT | DRINK | TALK | WALK | SIL | OTHER | TOTAL |
|---|---|---|---|---|---|---|---|
| Total | 119 | 27 | 27 | 59 | 332 | 8 | 572 |
| [hrs] | | | | | | | |

A typical experimental for a male subject resulted in the following detection rates:

False Negative: 3.7

False Positive: 3.8%

This indicates that 3.7% of the segments labeled as “eating” were recognized as “non-eating” segments and 3.8% of “non-eating” segments were recognized as “eating” segments. The confusion matrix shown below (Table 5) was utilized in order to look at the performance of the recognizer in more detail. Although optimized for eating detection, internally the recognizer is also modeling other segments such as DRINK, SIL (silence), SPEAK, and WALK). Each row shows how a given class was recognized. For example, 0.2% of EAT segments were recognized as DRINK, 96.3% as EAT, 2.9% as SIL, 0.3% as SPEAK, and 0.2% as WALK. Table 6 shows the results in seconds.

TABLE 5
Confusion Matrix [REF\HYP] (in percent)

| | | | | | | |
|---|---|---|---|---|---|---|
| DRINK | 51.8 | 7.5 | 39.5 | 0.5 | 0.4 | 100 |
| EAT | 0.2 | 96.3 | 2.9 | 0.3 | 0.2 | 100 |
| SIL | 2.8 | 4.1 | 88.8 | 2.2 | 1.7 | 100 |
| SPEAK | 0.3 | 0.7 | 4.0 | 93.7 | 1.0 | 100 |
| WALK | 0.1 | 1.2 | 5.7 | 1.9 | 90.9 | 100 |

TABLE 6
Confusion Matrix [REF\HYP] (in sec)

| | | | | | | |
|---|---|---|---|---|---|---|
| DRINK | 222 | 32 | 169 | 2 | 1 | 426 |
| EAT | 7 | 3752 | 114 | 13 | 7 | 3895 |
| SIL | 676 | 992 | 21381 | 540 | 408 | 24065 |
| SPEAK | 4 | 10 | 59 | 1385 | 14 | 1476 |
| WALK | 2 | 25 | 124 | 41 | 1965 | 2160 |
| ALL | 911 | 4811 | 21847 | 1981 | 2395 | 32022 |

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for monitoring eating behavior of a user comprising at least one sensor mounted on a head of the user, said at least one sensor being capable of detecting jaw muscle movement and sound while not occluding an ear canal of the user.

2. A device for monitoring eating behavior of a user comprising a sensor for detecting sounds generated by the user and an algorithm for applying formant analysis to sounds detected by said sensor to thereby identify sounds related to eating activity.

3. A device for monitoring eating behavior of a user comprising a sensor for detecting sounds generated by the user and an algorithm for collecting chewing sounds and categorizing said chewing sounds to thereby generate a log comprising data related to when, how long and how hard the user chews.

4. The device of claim 1, 2 or 3, wherein said at least one sensor includes a microphone and a tissue deformation sensor.

5. The device of claim 1, 2 or 3, wherein said at least one sensor is mountable behind an ear of the user.

6. The device of claim 1, 2 or 3, wherein said at least one sensor is a bubble microphone.

7. The device of claim 1, 2 or 3, further comprising an accelerometer for sensing body movement of the user.

8. The device of claim 1, 2 or 3, further comprising a processing unit being designed for processing input from said at least one sensor and extracting data related to an eating activity of the user.

9. The device of claim 8, wherein said processing unit is further designed for comparing said data related to an eating activity of the user to baseline data of the user as stored by said processing unit.

10. The device of claim 9, wherein the device is designed such that said baseline data of the user can be dynamically altered according to input from said processing unit or from an external source.

11. A device for monitoring eating behavior of a user comprising at least one sensor for monitoring sound and/or jaw movement of the user and a processing unit being designed for processing input from said at least one sensor and extracting data related to an eating activity of the user.

12. The device of claim 11, wherein said processing unit is further designed for comparing said data related to an eating activity of the user to baseline data of the user as stored by said processing unit.

13. The device of claim 12, wherein the device is designed such that said baseline data of the user can be dynamically altered according to input from said processing unit or from an external source.

14. A method of generating an eating log for a user comprising:
(a) monitoring eating behavior of a user over a time period and detecting eating activity;
(b) classifying said eating activity of the user over said time period;
(c) generating a log of said eating activity.

15. The method of claim 14, wherein said log includes data related to a duration of eating, rate of eating, food mass consumed, volume consumed and number of bites, chews and/or swallows over said time period.

16. The method of claim 14, wherein said log includes data related to a sequence of bites, chews and/or swallows over said time period.

17. The method of claim 14, wherein said log includes data related to when, how long and how hard the user chews.
